(19) United States
(12) Patent Application Publication
(10) Pub. No.: US 2024/0148647 A1
(43) Pub. Date: May 9, 2024

(54) AN EFLORNITHINE COMPOSITION FOR INHIBITING HAIR GROWTH

(71) Applicant: NAVIN SAXENA RESEARCH AND TECHNOLOGY PRIVATE LIMITED, Mumbai (IN)

(72) Inventors: Kunal SAXENA, Mumbai (IN); Navin SAXENA, Mumbai (IN)

(21) Appl. No.: 18/547,025

(22) PCT Filed: Feb. 19, 2022
(86) PCT No.: PCT/IN2022/050148

§ 371 Date: Aug. 18, 2023

(30) Foreign Application Priority Data
Feb. 20, 2021 (IN) 202121007200

(51) Int. Cl. A61K 9/06; A61K 9/00; A61K 31/198; A61K 47/44; A61P 17/00

(52) U.S. Cl. CPC A61K 9/06; A61K 9/0014; A61K 31/198; A61K 47/44; A61P 17/00

(57) ABSTRACT

The present invention discloses a topical cream composition of eflornithine or its pharmaceutically acceptable salts or prodrugs thereof with lavender oil as penetration enhancer and other excipients, which is effective to reduce the rate of skin hair growth and studies pertaining to pharmacodynamic behaviour thereof on hair growth rate. The invention further discloses a process for preparing a topical composition in the form of oil-in-water emulsion based cream composition which comprises eflornithine or its pharmaceutically acceptable salts or prodrugs thereof with a penetration enhancer and other excipients.

### FIELD OF THE INVENTION

The present invention relates to a new topical composition for inhibiting hair growth. It also relates to a method for inhibiting hair growth by applying a topical composition of eflornithine hydrochloride to the skin.

### BACKGROUND OF THE INVENTION

Hypertrichosis and hirsutism are terms generally used for excessive hair growth in different parts of the body. Hypertrichosis is also known as “werewolf syndrome” which can be generalized (present all over the body) or localized (restricted to certain areas) in nature. It might be congenital or acquired in later life stages. Hair follicle is the most self-regenerating and proliferating mini organ and passes through a cyclic phase of growth (anagen), regression (catagen) and resting (telogen).

Topical drug delivery can be defined as application of drug through skin to directly treat or cure the skin disorders. These systems are generally used for local skin infection like fungal infection or in place where other routes of the drug administration fails. Topical dosage forms are generally confined to a small area anywhere in the body through ophthalmic, rectal, vaginal and skin as a route. Skin is one of the most easily accessible organs of human body. Skin of an average adult body covers a surface of about 2 m² and receives around one-third of the blood circulating through the body.

Even though the intact skin is less permeable than other tissues, many of the drug molecules do penetrate the skin to some degree at relatively slow rates. The penetration of the drug and other substances depends mainly on the physiochemical properties of the penetrating agent, the condition of the skin and the nature of the vehicle. Commercially available drugs that are applied topically provide local action includes anti-fungal, anti-septic, anti-inflammatory agents as well as skin emollients for the protective effects.

Topically applied drugs can diffuse through the skin through hair follicles, sweat glands or sebaceous glands but permeation through multiple lipid bilayers of stratum corneum is a dominant pathway though the rate is very slow. Hirsutism is defined as the presence of terminal coarse hairs in females in a male-like distribution. It affects around 5-10% of women and is a common presenting complaint in the dermatological out-patient department (OPD) for cosmetic reasons. It is not only imperative to identify the cause of hirsutism but also important to know how to recommend the right treatment based on the main causative factor.

A topical cream composition of eflornithine hydrochloride (Figure (a)) can be applied to treat facial hirsutism. Chemically, eflornithine hydrochloride is (+)-2-(difluoromethyl) ornithine monohydrochloride monohydrate, (DEMO hydrochloride or Eflornithine) an irreversible inhibitor of ornithine decarboxylase (ODC), with the empirical formula C₆H₁₂F₂N₂O₂.HCl.H₂O, having a molecular weight of 236.65 and the following structural formula:

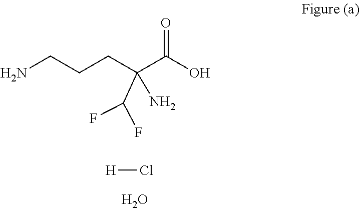

Anhydrous eflornithine hydrochloride has an empirical formula C₆H₁₂F₂N₂O₂.HCl and a molecular weight of 218.65. Eflornithine hydrochloride is used to treat excessive facial hair growth in women. Eflornithine is the first topical drug approved by FDA in July 2000, for the treatment of unwanted facial hair and is commercially offered by SkinMedica, Inc. under the brand name Vaniqa® in the form of a cream. Vaniqa® cream is containing 13.9% (139 mg/g) of anhydrous eflornithine hydrochloride in the form of eflornithine hydrochloride monohydrate (150 mg/g). The other ingredients include ceteareth-20, cetearyl alcohol, dimethicone, glyceryl stearate, methylparaben, mineral oil, PEG-100 stearate, phenoxyethanol, propylparaben, stearyl alcohol and water. Besides being a non-mechanical and non-cosmetic treatment, eflornithine is the only non-hormonal and non-systemic prescription option available for women who suffer from facial hirsutism.

Eflornithine hydrochloride is a water-soluble, crystalline solid, which is hygroscopic and susceptible to decomposition.

Eflornithine hydrochloride is prone to degradation when formulated with conventional pharmaceutical excipients into semi-solid dosage forms. Though exact mechanism of degradation has been fully elucidated, literature and patent information seem to indicate that decarboxylation is the possible mechanisms of degradation. The main degradation product is Ornithine.

The generic Eflornithine hydrochloride cream 13.9% was prepared by means of standard two-phase blending process forming an oil-in-water emulsion, further followed by homogenization using a homogenizer to ensure uniform dispersion and size reduction of ingredients with low speed mixing to remove air pockets in the dosage form. Time, temperature and mechanical energy input are the three major variables in the manufacturing of generic Eflornithine hydrochloride cream 13.9% preparation.

Vaniqa® is believed to inhibit the enzyme ornithine decarboxylase, an enzyme which is necessary for the synthesis of polyamines, which play an important role in cell growth and proliferation in the hair follicles. Percutaneous absorption in women when 15% cream was applied twice daily to a shaved 50 cm² area of skin under the chin was less than 1% and excreted unmetabolized. The onset of improvement according to the package insert for the product was seen in as little as four to eight weeks of treatment. The condition may return to pre-treatment levels within eight weeks after discontinuance of the treatment.

In U.S. Pat. No. 4,720,489 it is disclosed that the topical application of a composition comprising an ornithine decarboxylase (“ODC”) inhibitor that will inhibit hair growth, including beard hair growth. A particularly advantageous ODC inhibitor for use in this application is 2-(difluoromethyl)-2,5-diaminopentanoic acid, also identified as alpha-(difluoromethyl)-ornithine (“DFMO”). This patent demonstrates the effectiveness of ODC inhibitors for inhibiting hair growth by measuring changes in flank organ hair mass in adult male hamsters treated with ethanol solutions of such compounds. The patent also generally proposes the possible incorporation of ODC inhibitors in cosmetic formulations such as skin lotions and creams, but the effectiveness of such proposed formulations and their stability and aesthetic attributes are unknown. Since DFMO is a highly ionic material, it would tend to destabilize emulsion systems and would be difficult to formulate in such systems, particularly at higher dosage levels necessary to achieve maximum efficacy.

The Indian patent IN218050 (234/DELNP/2004) describes a topical composition for the delivery of one or more than one ornithine decarboxylase inhibitor applied to an area of the skin containing hair follicles wherein the composition comprising an ornithine decarboxylase inhibitor selected from the group consisting of 2-(difluoromethyl)-2,5-diaminopentanoic acid, α-ethynyl ornithine, 6-heptyne-2,5-diamine, 2-methyl-6-heptyne-2,5-diamine, pharmaceutically acceptable salts, hydrates, optical enantiomers, and racemic mixtures thereof, an enhancer is selected from the group consisting of poloxamer 407, steareth-20/steareth-100, poloxamer 185 and N, N-dimethyl dodecylamine N-oxide.

Excessive hair may cause cosmetic embarrassment, resulting in a significant emotional burden, particularly if extensive (Trueb R M. AM J ClinDermatol, 2002, 25 3(9): 617-27). There are several mechanical methods such as plucking, shaving and chemical methods such as waxing and thioglycolate treatment which are available for removal of excessive hairs.

An animal study was carried out with C57BL/6 mice for examining the physiological actions during different hair cycle phases of growth. Each experimental group was composed of 3 to 4 mice. Hair in the lower dorsal skin of anesthetized mice was either trimmed using an electric clipper, plucked using GiGi® Honee warm wax or chemically removed using Nair® lotion. The skin area where the hair removed was then treated with the eflornithine hydrochloride 13.9% cream (˜50 mg per mouse per treatment) using a spatula two times a day in an interval of at least 8 hours for a maximum period of 36 days.

A group of mice whose hair in the application site was trimmed using a clipper were also treated with the microneedle roller every time before the application of eflornithine cream as previously described (Amit Kumar, Youssef W. Naguib, Yan-Chun Shi and Zhengrong Cui; A method to improve the efficacy of topical eflornithine hydrochloride cream; Drug delivery. 2016 June; 1495-1501).

In control groups, the hair in mouse dorsal skin was removed by trimming, plucking or chemical depilation with Nair® lotion, but the area was not treated with the eflornithine cream. The hair re-growth was evaluated by taking digital photographs of the mouse skin areas for a maximum period of 36 days after the first application of the eflornithine cream. Eflornithine does not remove hair, but rather inhibits or actually slows down hair re-growth (Azziz 2003).

In these studies, the results showed that in mice whose hair in the dorsal skin area was removed by plucking or chemical depilation without further treatment with the eflornithine cream, significant hair growth was noticeable within 12 days after hair removal. However, in mice whose hair in the dorsal skin was trimmed, with or without further treatment with microneedles and applied with eflornithine cream, apparent hair re-growth was not observed until the 28^{th} day following hair removal. But in all groups, treatment with the eflornithine cream significantly inhibited hair re-growth. Treatment with the eflornithine cream following pre-treatment with microneedles was the most effective in inhibiting hair re-growth.

In the result, it was shown that pre-treatment of mouse skin with microneedles before topically applying eflornithine cream significantly enhanced eflornithine ability to inhibit hair growth. But the microneedle pre-treatment may causes skin irritations like swelling, discomfort at the site, redness, dryness and flaking of skin. Moreover the pre-treatment with microneedles before topically applying eflornithine cream shows the same efficacy as compared to the application of Eflornithine hydrochloride cream with penetration enhancer directly on the skin of mice.

However, there are several drawbacks associated with such methods some of which causes dryness, scaling, irritation and skin aberration. Thus, extensive research is being carried out specifically for the development of formulations containing Eflornithine which could provide better and effective treatment in retarding hair growth.

### SUMMARY OF THE INVENTION

According to one aspect, the invention describes the composition of eflornithine cream with penetration enhancer for enhancing the effectiveness of eflornithine in retarding hair growth over a concentration range of eflornithine.

The present invention further provides a study of pharmacodynamics behaviour of Eflornithine hydrochloride cream with penetration enhancer. The invention successfully demonstrated that Eflornithine hydrochloride cream with lavender oil as penetration enhancer is effective with the application of two times a day at an interval of at least 8 hours with in an animal study carried out in C57BL6 mice, in order to evaluate the efficacy of the invented composition.

According to another aspect, the invention provides Eflornithine hydrochloride cream formulation which is effective in preventing hair growth as compared to untreated control mice.

According to another aspect, Lavender oil is used in the cream formulation which provides a fragrance to Eflornithine hydrochloride cream formulation for better acceptance of the patient's aspect.

According to another aspect, Lavender oil in the cream composition serves to prevent scarring of the skin by promoting cell and tissue growth. Lavender oil has antibiotic, antioxidant, and anti septic properties.

According to another aspect, the invention wherein about 1 to 2000 micrograms of Eflornithine is applied per square centimeter of skin.

According to another aspect, the invention involves a method of inhibiting mammalian hair growth by applying an effective amount of the cream composition comprising Eflornithine hydrochloride and Lavender oil to the skin. The cream composition shows better results than the marketed composition, named V an i qa® .

### BRIEF DESCRIPTION OF FIGURES

FIG. 1 illustrates the results of a test with 0% of eflornithine and 4.0% of lavender oil applied on the lower dorsal skin of anesthetized mice by which the hair area was removed by waxing method. In the control group, the hair was removed by waxing method and not treated with eflornithine and lavender oil. The hair regrowth was measured by taking digital photographs and compared with that of the control group. Referring to FIG. 1 no significant hair growth was observed on 5^{th} day, some hair growth was observed on 11^{th} day and full hair growth was observed on 18^{th} day. The results show that formulation with Lavender oil alone can't show the hair growth inhibition activity.

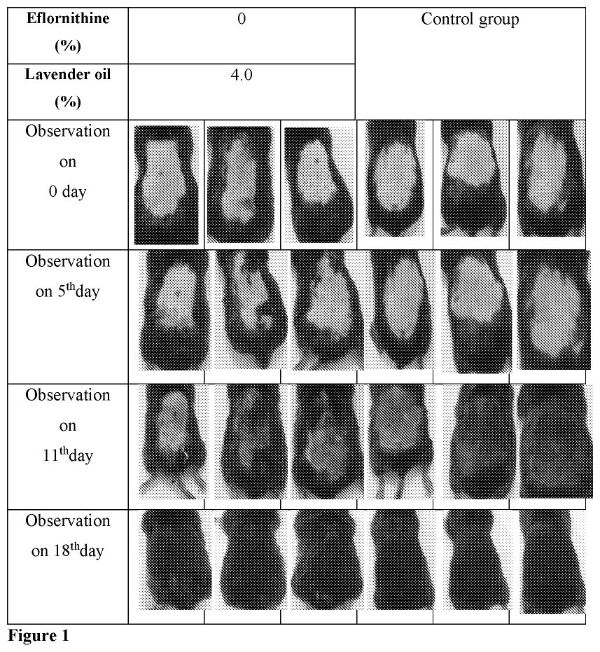

FIG. 2 illustrates the results of a test with 3.0% of eflornithine and 0.1% of lavender oil applied on the lower dorsal skin of anesthetized mice by which the hair area was removed by waxing method. In the control group, the hair was removed by waxing method and not treated with eflornithine and lavender oil. The hair regrowth was measured by taking digital photographs and compared with that of the control group. Referring to FIG. 2 no significant hair growth was observed on 5^{th} day, some hair growth was observed on 11^{th} day and full hair growth was observed on 18^{th} day. The results show that formulation with 3.0% of eflornithine and 0.1% of lavender oil is not sufficient to inhibit hair growth. Therefore the concentration of Lavender oil has been increased.

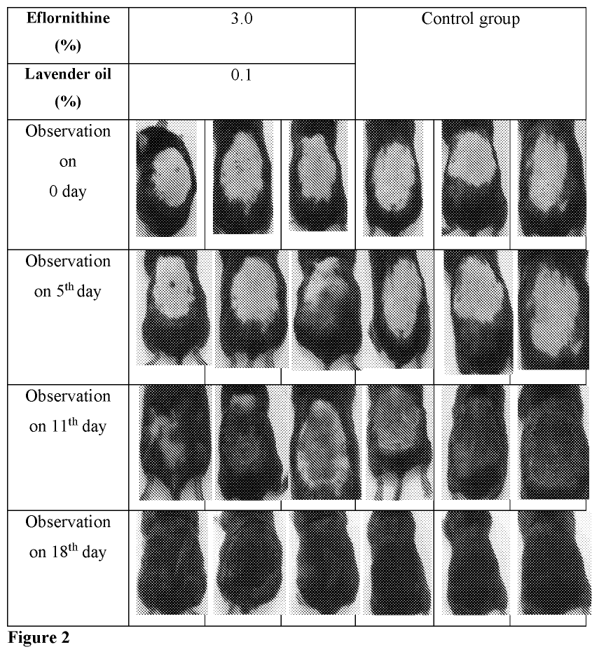

FIG. 3 illustrates the results of a test with 3.0% of eflornithine and 4.0% of lavender oil applied on the lower dorsal skin of anesthetized mice by which the hair area was removed by waxing method. In the control group, the hair was removed by waxing method and not treated with eflornithine and lavender oil. The hair regrowth was measured by taking digital photographs and compared with that of the control group. Referring to FIG. 1 no significant hair growth was observed on 5^{th} day, slight hair growth was observed on 11^{th} day and full hair growth was observed on 18^{th} day. The results show that the formulation with 3.0% of eflornithine and 4.0% of lavender oil is good enough to inhibit hair growth till 11^{th} day.

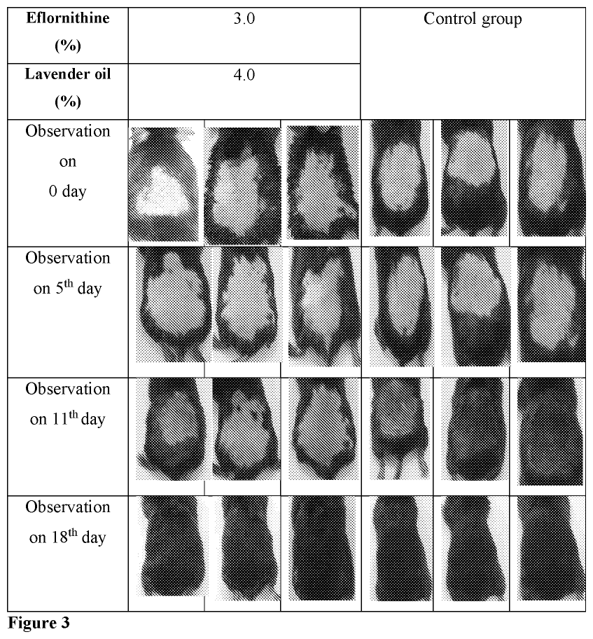

FIG. 4 illustrates the results of a test with 5.0% of eflornithine and 4.0% of lavender oil applied on the lower dorsal skin of anesthetized mice by which the hair area was removed by waxing method. In the control group, the hair was removed by waxing method and not treated with eflornithine and lavender oil. The hair regrowth was measured by taking digital photographs and compared with that of the control group. Referring to FIG. 1 no significant hair growth was observed on 5^{th} day, slight hair growth was observed on 11^{th} day and full hair growth was observed on 18^{th} day. The results show that the formulation with 5.0% of eflornithine and 4.0% of lavender oil is good enough to inhibit hair growth till 11^{th} day.

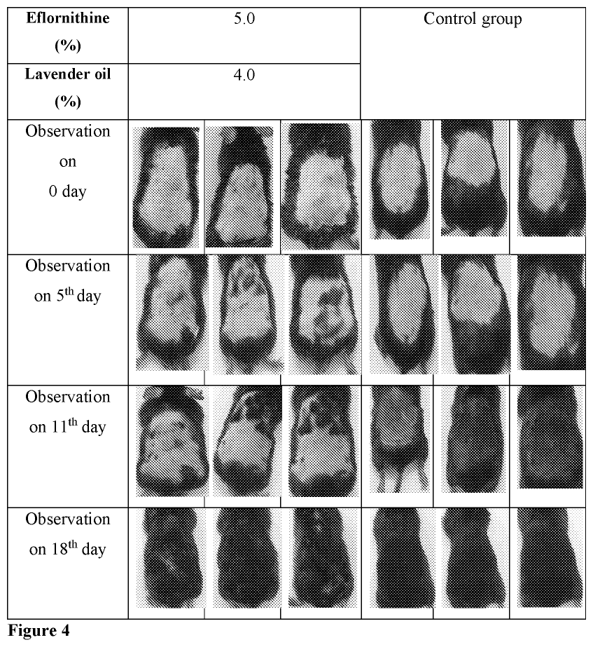

FIG. 5 illustrates the results of a test with 10.0% of eflornithine and 4.0% of lavender oil applied on the lower dorsal skin of anesthetized mice by which the hair area was removed by waxing method. In the control group, the hair was removed by waxing method and not treated with eflornithine and lavender oil. The hair regrowth was measured by taking digital photographs and compared with that of the control group. Referring to FIG. 1 no significant hair growth was observed on 5^{th} day, slight hair growth was observed on 11^{th} day and full hair growth was observed on 18^{th} day. The results show that the formulation with 10.0% of eflornithine and 4.0% of lavender oil is good enough to inhibit hair growth till 11^{th} day.

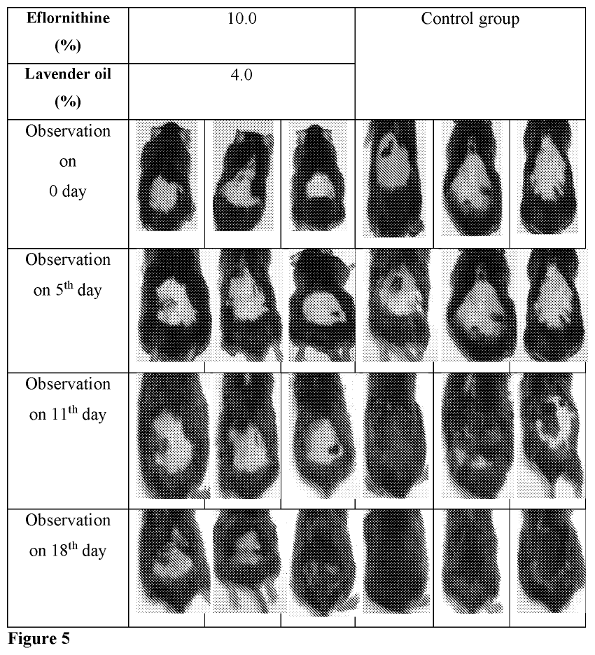

FIG. 6 illustrates the results of a test with 13.9% of eflornithine and 0.1% of lavender oil applied on the lower dorsal skin of anesthetized mice by which the hair area was removed by waxing method. In the control group, the hair was removed by waxing method and not treated with eflornithine and lavender oil. The hair regrowth was measured by taking digital photographs and compared with that of the control group. Referring to FIG. 1 no significant hair growth was observed on 5^{th} day, slight hair growth was observed on 11^{th} day and full hair growth was observed on 18^{th} day. The results show that the formulation with 13.9% of eflornithine and 0.1% of lavender oil is not sufficient enough to inhibit hair growth after 11^{th} day.

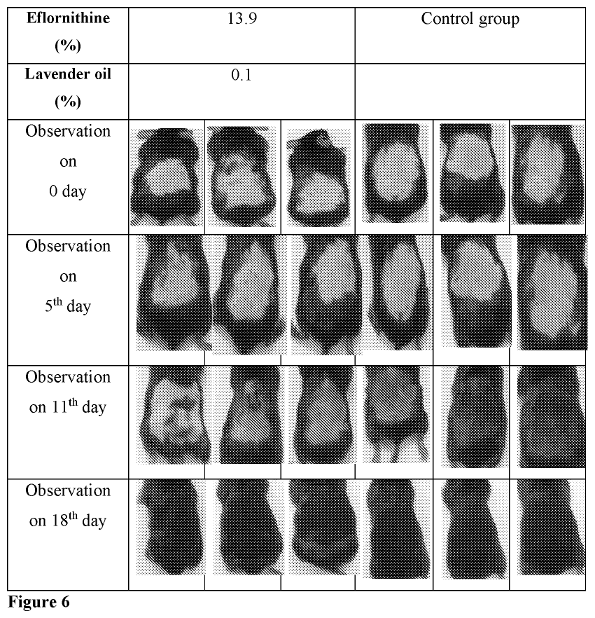

FIG. 7 illustrates the results of a test with 13.9% of eflornithine and 4.0% of lavender oil applied on the lower dorsal skin of anesthetized mice by which the hair area was removed by waxing method. In the control group, the hair was removed by waxing method and not treated with eflornithine and lavender oil. The hair re-growth was measured by taking digital photographs and compared with that of the control group. Referring to FIG. 1 no significant hair growth was observed on 5^{th} day, 11^{th} day and 18^{th} day. The results show that the formulation with 13.9% of eflornithine and 4.0% of lavender oil has shown best hair inhibiting capacity and it controls the hair growth till 18^{th} day.

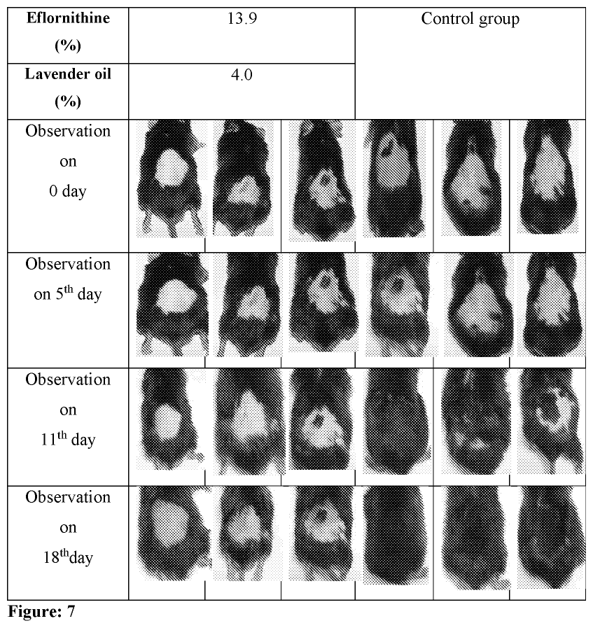

FIG. 8 illustrates the results of a test with 13.9% of eflornithine applied on the lower dorsal skin of anesthetized mice by which the hair area was removed by waxing method. In the control group, the hair was removed by waxing method and not treated with eflornithine. The hair re-growth was measured by taking digital photographs and compared with that of the control group. Referring to FIG. 8 no significant hair growth was observed on 5^{th} day, some hair growth was observed on 11^{th} day and full hair growth was observed on 18^{th} day. The results show that formulation with 13.9% of eflornithine is not sufficient to inhibit hair growth upto 18 days. Therefore Lavender oil is needed as permeation enhancer for eflornithine for inhibiting the hair growth.

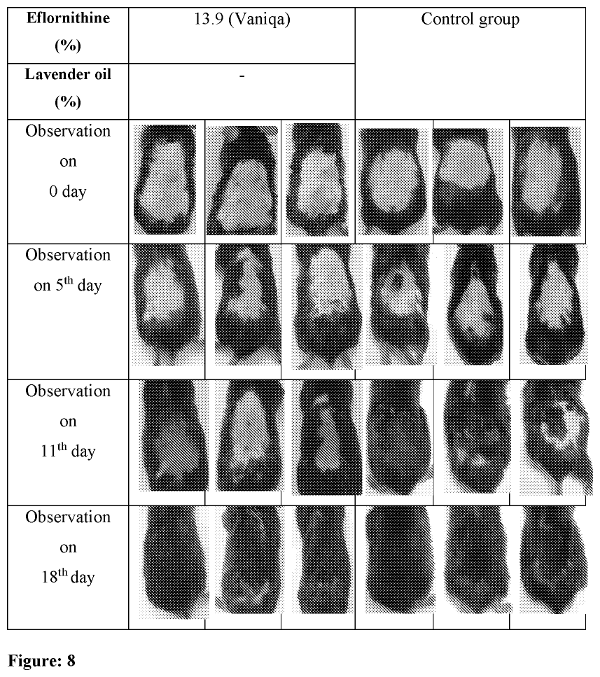

### DESCRIPTION OF INVENTION

The present invention provides a topical cream compositions comprising Eflornithine along with a penetration enhancer, useful for the treatment for Hirsutism. Hirsutism is defined as the presence of terminal coarse hairs in females in a male-like distribution which can be treated by topically applying the Eflornithine cream composition on the skin. In particular, the topical Eflornithine hydrochloride irreversibly inhibits skin ODC (Ornithine decarboxylase) activity. This enzyme is necessary for the synthesis of polyamines.

As used herein, the term “penetration enhancer” refers to an agent that improves the transport of molecules such as an active agent (e.g., a medicine) into or through the skin.

The term “salts,” as used herein, includes but is not limited to sodium, potassium, calcium, ammonium, manganese, copper, and/or magnesium salts of a given compound.

The term “prodrug”, means a pharmacological inactive derivative of a drug molecule that is capable of releasing the parent molecule quantitatively, due to enzymatic or spontaneous reaction in the body.

The term “hair,” as used herein, includes hair on any part of the body.

Accordingly, the present invention provides a topical cream composition of eflornithine or its pharmaceutically acceptable salts or prodrugs thereof, effective to reduce the rate of skin hair growth, wherein the composition comprises:
a) Eflornithine at a concentration of about 3% w/w to 13.9% w/w of total composition,
b) a penetration enhancer at a concentration of about 0.1% w/w to about 25.0% w/w of total composition,
c) an oil in water emulsifier at a concentration of about 1.0% w/w to about 12.0% w/w of total composition,
d) an emulsion stabiliser at a concentration of about 3.75% w/w to about 12.0% w/w of total composition,
e) an emulsifier at a concentration of about 1.0% w/w to about 12.0% w/w of total composition,
f) a defoaming agent at a concentration of about 1.0% w/w to about 9.0% w/w of total composition,
g) a moisturizing agent and a buffering agent at a concentration of about 0.01% w/w to about 2.0% w/w of total composition,
h) a preservative at a concentration of about 0.1% w/w to about 0.8% w/w of total composition and
i) a topical carrier.

In an embodiment, the eflornithine is present in the form of eflornithine hydrochloride.

The topical composition provided according to the present invention comprises; wherein,
a. the penetration enhancer is as an essential oil selected from angelica oil, lavender oil, cyperus oil, cinnamon oil, basil oil, jasmine oil and clove oil, preferably the essential oil is lavender oil;
b. the oil in water emulsifier is selected from glyceryl stearate and macrogol stearate, sorbitan stearate, poly glyceryl oleate, sorbitan rnonooleate and lanolin;
c. the emulsion stabiliser is selected from cetostearyl alcohol and macrogol ceto stearyl ether, soy lecithin, sodium phosphate, mono and diglycerides and sodium stearoylactylate;
d. the emulsifier is selected from stearyl alcohol, tragacanth, sodium lauryl sulphate and sodium dioctylsulfosuccinate.
e. the defoaming agent is selected from polydimethylsiloxane, sulfonated oils and mineral oils,
f. the moisturizing and a buffering agent is selected from glycerine, citric acid anhydrous, phosphoric acid, lactic acid, glycolic acid, citric acid, malic acid, tartaric acid, gluconic acid, or gluconolactone sodium hydroxide and monobasic sodium phosphate,
g. the preservative selected from parabens such as methyl paraben, propyl paraben, phenoxyethanol, ethylhexylglycerin and propylene glycol and
h. the topical carrier is selected from purified water or light liquid paraffin.

In one embodiment of the present invention, the invention provides a method for the development of Eflornithine Hydrochloride Cream which is equivalent strength to Vaniqa® cream which contains 15% w/w eflornithine hydrochloride monohydrate, corresponding to 11.5% w/w anhydrous eflornithine (EU), respectively 13.9% w/w anhydrous eflornithine hydrochloride (U.S.), in a cream for topical administration.

In other embodiment of the present invention, the invention provides a method for the development of Eflornithine Hydrochloride cream ranging from a concentration of 3% w/w to 13.9% w/w of Eflornithine hydrochloride.

In a preferred embodiment of the invention to topical composition comprises lavender oil in the concentration of 0.1% w/w to about 25.0% w/w as penetration enhancer to the skin for retarding hair growth and also used as a fragrant in Eflornithine Hydrochloride Cream 13.9% w/w composition.

Another embodiment of the invention, Lavender oil when combined with Eflornithine Hydrochloride contributes to the inhibition of hair follicle activity, inhibit the growth of hair follicles.

The emulsion stabiliser is selected from the group consisting of cetostearyl alcohol and macrogol ceto stearyl ether at a concentration of about 3.75% w/w to about 12.0% w/w of total composition.

The emulsifier is stearyl alcohol present at a concentration of about 1.0% w/w to about 12.0% w/w of total composition.

The defoaming agent is polydimethylsiloxane at a concentration of about 1.0% w/w to about 9.0% w/w of total composition.

The moisturizing and buffering agent is selected from citric acid anhydrous at a concentration of about 0.01% w/w to about 2.0% w/w of total composition.

The preservative is selected from methyl paraben and propyl paraben at a concentration of about 0.1% w/w to about 0.8% w/w of total composition.

The topical composition according to the present invention is oil in water emulsion based cream.

In yet another embodiment, the invention provides a process for preparing topical composition of the eflornithine or its pharmaceutically acceptable salts or prodrugs thereof in the form of an oil in water emulsion based cream, effective to reduce the rate of skin hair growth, comprising the steps of:
a. preparing an oil phase by transferring glyceryl stearate and macrogol stearate, ceteostearyl alcohol and macrogol ceto stearyl ether, light liquid paraffin, stearyl alcohol and poly dimethyl siloxane in jacketed oil phase vessel,
b. adding methyl paraben and propyl paraben to the melted mass by continuous stirring at a temperature of 50° C. to 90° C.,
c. adding lavender oil slowly into the melted mass by continuous stirring,
d. preparing an aqueous phase by transferring exactly half dispensed quantity of purified water and heating to a temperature of 60° C. to 65° C. and citric acid is added with continuous stirring to get a clear solution,
e. The oil phase material is added to the aqueous phase by maintaining the temperature to 70° C. to 80° C.,
f. Preparing a drug solution phase by adding eflornithine hydrochloride monohydrate to the remaining half-quantity of purified water with continuous stirring,
g. Adding drug solution phase to oil-in-water phase with continuous stirring by maintaining the temperature to 40° C. to 80° C.,
h. Cooling to room temperature with continuous stirring till the cream congeals and maintaining the pH to 2 to 7 and
i. Filling the tubes with eflornithine hydrochloride cream obtained in step f) using automatic tube filling and sealing machine.

The topical composition provided according to the present invention comprising eflornithine effective to reduce the rate of skin hair growth observed no hair growth on the skin and which is effective with the application of two times a day at an interval of at least 8 hours for a period of 18 days to retard the skin hair growth.

In another preferred embodiment, the present topical compositions of eflornithine was evaluated for its efficacy in retarding the rate of hair growth. The composition of Eflornithine Hydrochloride Cream 13.9% provided according to the present invention (Topical formulation 7) has shown retardation in the rate of hair growth efficiently.

In another embodiment, the excipient-drug substance compatibility studies were conducted and each excipient grade was selected. The excipients functioning as emulsifier, stabiliser, de-foaming agent, moisturizing and buffering agent, preservatives, fragrance and vehicles were evaluated in the excipient compatibility study.

The stability of the drug was evaluated by measuring the amount of the degradation product and assay at each of the time of preparation from the initial time and respective times after stored at 40° C.±2° C. and 75% RH±5% RH for six months. About a judgement criterion (index for quality and assurance) for the cream composition was judged to be good in drug stability.

Stability trend of Eflornithine hydrochloride cream 13.9% w/w at the condition of 40° C.±2° C. and 75% RH±5% RH.

| Sr no | Test parameters | specification | Initial | 1 month | 3 months | 6 months |
|---|---|---|---|---|---|---|
| 1. | Description | White to off- | White to off- | White to off- | White to off- | White to off- |
| | | white cream | white cream | white cream | white cream | white cream |
| 2. | pH (10% aqueous | 3.0-5.0 | 3.4 | 3.4 | 3.4 | 3.0 |
| | dispersion) | | | | | |
| 3. | Assay Eflornithine | 90-110% | 102.39 | 101.87 | 102.19 | 103.41 |
| | HCl | | | | | |
| | Methyl paraben | 90-110% | 99.28 | 98.04 | 97.03 | 95.96 |
| | Propyl paraben | 90-110% | 96.57 | 96.74 | 95.22 | 93.55 |
| 4. | RS (by TLC) | L-ornithine: | Complies | Complies | Complies | Complies |
| | | NMT 0.5% w/w | | | | |

The creams or cream bases which can be prepared in accordance with the process of the invention are semisolid emulsions of oil-in-water type. The cream include from about 10% to about 90% by weight of an oil phase, from about 90% to about 10% by weight of an aqueous phase. The oil phase generally comprises from about 0.1% w/w to about 25.0% w/w of an oil material such as essential oil. Other ingredients which can be present in the oil phase include, for example, emulsifiers, emulsion stabiliser, defoaming agent, moisturizing and buffering agent, preservative and purified water.

The details of the excipients which were formulated with Eflornithine Hydrochloride Cream 13.9% of the present invention are:

### Penetration Enhancers:

a. Essential Oil:

Essential oils and their constituents are used in the transdermal drug delivery system as a skin permeation enhancer. Essential oils and their volatile constituents can penetrate through the skin as well as enhance penetration of different drug from topical formulation into the lower skin layers using different mechanisms of action based on (1) disintegration of the highly ordered intercellular lipid structure between corneocytes in stratum corneum, (2) interaction with intercellular domain of protein, which induces their conformational modification, (3) increase the partitioning of a drug. After application to the skin, essential oils and their components are rapidly metabolized, not accumulated in the organism and fast excreted from the body and can be successfully used as safe penetration enhancers. (Anna Herman, Andrzej P Herman; Essential oils and their constituents as skin penetration enhancer for transdermal drug delivery: a review).

The essential oil is selected from the group consisting of angelica oil, lavender oil, cyperus oil, cinnamon oil, basil oil, jasmine oil and clove oil. The preferred essential oil is Lavender oil, a pale-yellow liquid with a characteristic slightly camphoraceous odour, is obtained from Lavendula angustifolia Mill. (syn. L. officinalis Chaix, L. vera D.C.). Lavender oil is used for decreasing hair growth in young women affected by hirsutism in the light of their possible anti-androgenic effects. This treatment could represent a safe, economic and practical instrument in the cure of hirsutism. (G. Tirabassi, L. Giovannini, F. Paggi, G. Panin, F. Panin, R. Papa, M. Boscaro and G. Balercia, J. Endocrinol. Invest. 36: 50-54, 2013). Essential oils including lavender oil may be preferred over the traditionally used synthetic materials as safe and suitable penetration enhancer to promote the percutaneous absorption of hydrophilic and lipophilic drugs from topical formulation into the lower skin layers (Anna Herman and Andrzej P. Herman: Essential oils and their constituents as skin penetration enhancer for transdermal drug delivery: a review; August 2014, 473-485). Lavender oil is also used to enhance antifungal efficacy and stability of clotrimazole by formulating as emulgels (combined topical dosage forms of gel and emulsion to treat chronic skin diseases like fungal infections, acne and psoriasis) using different gelling agents and lavender oil as oil phase. The inclusion of lavender oil has supplemented with the antifungal action of clotrimazole against Trichophytonrubrum. (VaishaliPotnis, Mahesh Khot, Mangesh Kahane, Sindhu Mohite, Pradnya Bhongale, Suchita Dhamane: Formulation of gellified emulsions of Clotrimazole using essential oil from Lavendula angustifolia Miller; 2014, 2321-3310).Lavender and Tea tree oils are used in women suffering from mild idiopathic hirsutism (G. Tirabassi, L. Giovannini, F. Paggi, et al; Possible efficacy of Lavender and Tea tree oils in the treatment of young women affected by mild idiopathic hirsutism). Lavender oil can be used for the topical administration as a penetration enhancer to the skin as well as can be used for the treatment of mild idiopathic hirsutism for inhibiting hair growth.

b. Transcutol®:

Transcutol® or diethylene glycol monoethyl ether is a liquid which has a long history of use in cosmetic and over-the-counter topically applied products. Transcutol® has been applied in several commercial preparations and is used in many studies as the main ingredient of formulations. It has been used in formulations as a co-surfactant and a penetration enhancer. It is frequently utilized in topical formulations (dermal drug delivery systems), transdermal delivery systems, and ocular and also intranasal delivery systems. (Yousef Javadzadeh, Khosro Adibkia and Hamed Hamishekar: Transcutol® (Diethylene Glycol Monoethyl Ether): A Potential Penetration Enhancer; January 2015, 194-205).

c. Dimethyl Sulphoxides (DMSO):

This is one of the earliest and most widely studied penetration enhancers. It is a powerful aprotic solvent which hydrogen bonds with itself rather than with water. It is colourless, odourless and is hydroscopic and is often used in many areas of pharmaceutical sciences as a “universal solvent”. DMSO alone has been applied topically to treat systemic inflammation. DMSO works rapidly as a penetration enhancer. (Inayat Bashir Pathan, C. Mallikarjuna Setty; Chemical Penetration Enhancers for Transdermal Drug Delivery Systems; 173-179).

d. Polyethylene Glycol (PEG):

PEG was shown to have significant effects on drug penetration when skin structures were hygroscopically manipulated (Sarpotdar et al., 1986). Preliminary research has also shown that fatty acids linked to structurally configured polymers act as penetration enhancers, modifying the barrier properties of the stratum corneum and allowing migration of drugs in human skin (Martin, 1993). (Sherry Jung: Novel Compounds For Skin Penetration Enhancement; 25-30).

### Oil-in-Water Emulsifier:

The oil-in-water emulsifier is selected from the group consisting of glyceryl stearate and macrogol stearate, sorbitan stearate, polyglyceryloleate, lecithin, sorbitanmonooleate and lanolin. The preferred oil-in-water emulsifier is Macrogol stearate and Glycerylstearate. This is an acid-stable self-emulsifying agent. The primary use of this product is as an emulsifier for creams and lotions that are rich in non-polar oils and waxes. Because these are non-ionic emulsifiers, they will tolerate the addition of acidic actives and salts. In addition, emulsions made with them can be pH adjusted to between 4.5 and as high as 9. The concentration of Macrogol stearate and Glyceryl stearate to be used as emulsifier in anhydrous cream bases is from 1 to 12% w/w.

### Emulsion Stabiliser:

The emulsion stabiliser is selected from the group consisting of cetostearyl alcohol and macrogol cetostearyl ether, soy lecithin, sodium phosphate, mono and diglycerides and sodium stearoyl lactylate. The preferred emulsion stabiliser is cetostearyl alcohol and macrogol ceto stearyl ether which can be used in an amount of 3.75% w/w to 12.0% w/w. This is an extremely versatile non-ionic surface active agent which may be employed as primary or secondary oil-in-water emulsifiers, bodying agents or opacifiers, enabling the formulation of a variety of cosmetic and pharmaceutical systems. This grade produces emulsions of intermediate viscosity and is recommended for use in cosmetic and pharmaceutical creams. In common, the emulsions based on this material will tolerate high levels of electrolytes.

### Emusifier:

The emulsifier is selected from the group consisting of stearyl alcohol, tragacanth, sodium lauryl sulphate and sodium dioctyl sulfo succinate. The preferred emulsifier in the composition is stearyl alcohol which is present in about 1.0% w/w to about 12.0% w/w. Stearyl alcohol occurs as hard, white, waxy pieces, flakes, or granules with a slight characteristic odour and bland taste. Stearyl alcohol is used in cosmetics and topical pharmaceutical creams and ointments as a stiffening agent. By increasing the viscosity of an emulsion, stearyl alcohol increases its stability. Stearyl alcohol also has some emollient and weak emulsifying properties and is used to increase the water-holding capacity of ointments, e.g. petrolatum. It has also been investigated for use as a transdermal penetration enhancer.

### Defoaming Agent:

The defoaming agent is selected from the group consisting of polydimethylsiloxane, sulfonated oils and mineral oils. The preferred defoaming agent is polydimethylsiloxane which is present in about 1.0% w/w to about 9.0% w/w in the composition.

### Moisturizing and Buffering Agent:

This is selected from the group consisting of citric acid, phosphoric acid, sodium hydroxide and monobasic sodium phosphate. The preferred moisturizing and buffering agent is citric acid monohydrate which occurs as colourless or translucent crystals, or as a white crystalline, efflorescent powder. Citric acid (as either the monohydrate or anhydrous material) is widely used in pharmaceutical formulations and food products, primarily to adjust the pH of solutions in the concentration range of 0.01-2.0%.

### Preservatives:

a. Methyl Paraben:

Methylparaben occurs as colourless crystals or a white crystalline powder. Methylparaben is widely used as an antimicrobial preservative in cosmetics, food products, and pharmaceutical formulations; it may be used either alone or in combination with other parabens or with other antimicrobial agents. The parabens are effective over a wide pH range and have a broad spectrum of antimicrobial activity, although they are most effective against yeasts and moulds. It is used in the concentration range of 0.02-3.0% as antimicrobial preservatives in topical formulation.

b. Propyl Paraben:

Propyl paraben occurs as a white, crystalline, odourless, and tasteless powder. Propyl paraben is widely used as an antimicrobial preservative in cosmetics, food products, and pharmaceutical formulations. It may be used alone, in combination with other paraben esters, or with other antimicrobial agents. The parabens are effective over a wide pH range and have a broad spectrum of antimicrobial activity, although they are most effective against yeasts and moulds. It is used in the concentration range of about 0.01-0.6% as antimicrobial preservatives in topical formulation. Methyl paraben and propyl paraben and their sodium salts are frequently used in semisolids because of their better solubility in aqueous and oil phases, respectively.

c. Butylatedhydroxyanisole (BHA)

BHA is an antioxidant with some antimicrobial properties. It is used in a wide range of cosmetics, foods and pharmaceuticals. BHA is frequently used in combination with other antioxidants, particularly butylated hydroxyl toluene and alkyl gallates and with sequestrants or synergists such as citric acid. BHA is used in the concentration range of about 0.01 to 5.2% as an antioxidant in the topical formulation (Raymond C Rowe, Paul J Sheskey and Marian E Quinn: Handbook of Pharmaceutical Excipients 6^{th} Edition; 73-74).

d. Butylatedhydroxytoluene (BHT)

BHT is used as an antioxidant in cosmetics, foods ; and pharmaceuticals. Itis mainly used to delay or prevent the oxidative rancidity of fats and oils and to prevent loss of activity of oil-soluble vitamins. Butylated hydroxyl toluene has some antiviral activity and has been used therapeutically to treat herpes simplex labialis. (Raymond C Rowe, Paul J Sheskey and Marian E Quinn: Handbook of Pharmaceutical Excipients 6^{th} Edition; 75-76) BHT is used in the concentration range of 0.01-0.1%.

The present inventors have successfully developed the Eflornithine hydrochloride cream 13.9% w/w which is equivalent strength to Vaniqa® cream which contains 15% w/w eflornithine hydrochloride monohydrate, corresponding to 11.5% w/w anhydrous eflornithine (EU), respectively 13.9% w/w anhydrous eflornithine hydrochloride (U.S.), in a cream for topical administration. In addition inventor have also successfully developed the Eflornithine Hydrochloride cream ranging from a concentration of 3% w/w to 13.9% w/w of Eflornithine hydrochloride.

### EXAMPLES

Preparation of Eflornithine hydrochloride cream composition in the present invention:

| Sr No. | Ingredients | Function | % w/w |
|---|---|---|---|
| 1. | Eflornithine HCl monohydrate | Active | 3.0-13.9 |
| 2. | Glycerylstearate and | Oil in Water | 1.0-12.0 |
| | Macrogol stearate | Emulsifier | |
| | (Arlacel ™ 165) | | |
| 3. | Cetearyl alcohol and | Emulsion stabiliser | 3.75-12.0 |
| | Macrogol cetostearyl ether | | |
| | (Cosmo wax ™ D) | | |
| 4. | Light liquid paraffin | Vehicle | 1.0-3.0 |
| 5. | Stearyl alcohol | Emulsifier | 1.0-12.0 |
| 6. | Polydimethylsiloxane | Defoaming agent | 1.0-9.0 |
| | (Silicone fluid) or | | |
| | Dimethicone | | |
| 7. | Citric acid anhydrous | Moisturizing and | 0.01-2.0 |
| | | buffering agent | |
| 8. | Methyl paraben | Preservative | 0.1-1.8 |
| | (Methyl hydroxy benzoate) | | |
| 9. | Propyl paraben | Preservative | 0.1-1.8 |
| | (Propyl hydroxy benzoate) | | |
| 10. | Lavender oil | Penetration | 0.1-25.0 |
| | | enhancer | |
| | | and Fragrant | |
| 11. | Purified water | A topical carrier | q.s |
| 12. | Purified water (Part II)* | — | — |

*Compensated quantity of Purified water against the quantity of Eflornithine Hydrochloride Monohydrate.

### The Quantities of Each Component are Mentioned in the Example Section Given Below.

### Example 1

Preparation of Eflornithine hydrochloride cream composition in the present invention:

| | | | | |
|---|---|---|---|---|
| Sr No. | Ingredients | Function | mg/g | % w/w |

DRUG PHASE

| | | | | |
|---|---|---|---|---|
| 1. | Eflornithine HCl | Active | — | — |
| | monohydrate | | | |

OIL PHASE

| | | | | |
|---|---|---|---|---|
| 2. | Glycerylstearate and | Oil in Water | 50.0 | 5.0 |
| | Macrogol stearate | Emulsifier | | |
| | (Arlacel ™ 165) | | | |
| 3. | Cetearyl alcohol and | Emulsion | 75.03 | 7.50 |
| | Macrogol cetostearyl | stabiliser | | |
| | ether (Cosmowax ™ D) | | | |
| 4 | Light liquid paraffin | Vehicle | 30.0 | 3.0 |
| 5 | Stearyl alcohol | Emulsifier | 20.0 | 2.0 |
| 6. | Polydimethylsiloxane | Defoaming agent | 20.0 | 2.0 |
| | (Silicone fluid) or | | | |
| | Dimethicone | | | |
| 7. | Citric acid anhydrous | Moisturizing and | 1.30 | 0.13 |
| | | buffering agent | | |
| 8. | Methyl paraben | Preservative | 1.80 | 0.18 |
| | (Methyl hydroxy | | | |
| | benzoate) | | | |
| 9. | Propyl paraben | Preservative | 0.2 | 0.02 |
| | (Propyl hydroxy | | | |
| | benzoate) | | | |
| 10. | Lavender oil | Penetration | 40.0 | 4.0 |
| | | enhancer and | | |
| | | Fragrant | | |

WATER PHASE

| | | | | |
|---|---|---|---|---|
| 11. | Citric acid anhydrous | Moisturizing and | 1.30 | 0.13 |
| | | buffering agent | | |
| 12. | Purified water (Part I) | A topical carrier | 305.32 | 30.53 |
| 13. | Purified water (Part II) | | 455.05 | 45.51 |

### Example 2

Preparation of Eflornithine hydrochloride cream composition in the present invention:

| | | | | |
|---|---|---|---|---|
| Sr No. | Ingredients | Function | mg/g | % w/w |

DRUG PHASE

| | | | | |
|---|---|---|---|---|
| 1. | Eflornithine HCl | Active | 30.0 | 3.0 |
| | monohydrate | | | |

OIL PHASE

| | | | | |
|---|---|---|---|---|
| 2. | Glycerylstearate and | Oil in Water | 50.0 | 5.0 |
| | Macrogol stearate | Emulsifier | | |
| | (Arlacel ™ 165) | | | |
| 3. | Cetearyl alcohol and | Emulsion | 75.03 | 7.50 |
| | Macrogol cetostearyl | stabiliser | | |
| | ether (Cosmowax ™ D) | | | |
| 4. | Light liquid paraffin | Vehicle | 30.0 | 3.0 |
| 5. | Stearyl alcohol | Emulsifier | 20.0 | 2.0 |
| 6. | Polydimethylsiloxane | Defoaming agent | 20.0 | 2.0 |
| | (Silicone fluid) or | | | |
| | Dimethicone | | | |
| 7. | Citric acid anhydrous | Moisturizing and | 1.30 | 0.13 |
| | | buffering agent | | |
| 8. | Methyl paraben | Preservative | 1.80 | 0.18 |
| | (Methyl hydroxy | | | |
| | benzoate) | | | |
| 9. | Propyl paraben | Preservative | 0.2 | 0.02 |
| | (Propyl hydroxy | | | |
| | benzoate) | | | |
| 10. | Lavender oil | Penetration | 1.0 | 0.1 |
| | | enhancer and | | |
| | | Fragrant | | |

WATER PHASE

| | | | | |
|---|---|---|---|---|
| 11. | Citric acid anhydrous | Moisturizing and | 1.30 | 0.13 |
| | | buffering agent | | |
| 12. | Purified water (Part I) | A topical carrier | 305.32 | 30.53 |
| 13. | Purified water (Part II)* | | 464.05 | 46.41 |

*Compensated quantity of Purified water against the quantity of Eflornithine Hydrochloride Monohydrate

### Example 3

Preparation of Eflornithine hydrochloride cream composition in the present invention:

| | | | | |
|---|---|---|---|---|
| Sr No. | Ingredients | Function | mg/g | % w/w |

DRUG PHASE

| | | | | |
|---|---|---|---|---|
| 1. | Eflornithine HCl | Active | 30.0 | 3.0 |
| | monohydrate | | | |

OIL PHASE

| | | | | |
|---|---|---|---|---|
| 2. | Glycerylstearate and | Oil in Water | 50.0 | 5.0 |
| | Macrogol stearate | Emulsifier | | |
| | (Arlacel ™ 165) | | | |
| 3. | Cetearyl alcohol and | Emulsion | 75.03 | 7.50 |
| | Macrogol cetostearyl | stabiliser | | |
| | ether (Cosmowax ™ D) | | | |
| 4. | Light liquid paraffin | Vehicle | 30.0 | 3.0 |
| 5. | Stearyl alcohol | Emulsifier | 20.0 | 2.0 |
| 6. | Polydimethylsiloxane | Defoaming agent | 20.0 | 2.0 |
| | (Silicone fluid) or | | | |
| | Dimethicone | | | |
| 7. | Citric acid anhydrous | Moisturizing and | 1.30 | 0.13 |
| | | buffering agent | | |
| 8. | Methyl paraben | Preservative | 1.80 | 0.18 |
| | (Methyl hydroxy | | | |
| | benzoate) | | | |
| 9. | Propyl paraben | Preservative | 0.2 | 0.02 |
| | (Propyl hydroxy | | | |
| | benzoate) | | | |
| 10. | Lavender oil | Penetration | 40.0 | 4.0 |
| | | enhancer and | | |
| | | Fragrant | | |

WATER PHASE

| | | | | |
|---|---|---|---|---|
| 11. | Citric acid anhydrous | Moisturizing and | 1.30 | 0.13 |
| | | buffering agent | | |
| 12. | Purified water | A topical carrier | 305.32 | 30.53 |
| 13. | Purified water | | 425.05 | 42.51 |
| | (Part II)* | | | |

### Example 4

Preparation of Eflornithine hydrochloride cream composition in the present invention:

| | | | | |
|---|---|---|---|---|
| Sr No. | Ingredients | Function | mg/g | % w/w |

DRUG PHASE

| | | | | |
|---|---|---|---|---|
| 1. | Eflornithine HCl | Active | 50.0 | 5.0 |
| | monohydrate | | | |

OIL PHASE

| | | | | |
|---|---|---|---|---|
| 2. | Glycerylstearate and | Oil in Water | 50.0 | 5.0 |
| | Macrogol stearate | Emulsifier | | |
| | (Arlacel ™ 165) | | | |
| 3. | Cetearyl alcohol and | Emulsion | 75.03 | 7.50 |
| | Macrogol cetostearyl | stabiliser | | |
| | ether (Cosmowax ™ D) | | | |
| 4. | Light liquid paraffin | Vehicle | 30.0 | 3.0 |
| 5. | Stearyl alcohol | Emulsifier | 20.0 | 2.0 |
| 6. | Polydimethylsiloxane | Defoaming agent | 20.0 | 2.0 |
| | (Silicone fluid) or | | | |
| | Dimethicone | | | |
| 7. | Citric acid anhydrous | Moisturizing and | 1.30 | 0.13 |
| | | buffering agent | | |
| 8. | Methyl paraben | Preservative | 1.80 | 0.18 |
| | (Methyl hydroxy | | | |
| | benzoate) | | | |
| 9. | Propyl paraben | Preservative | 0.2 | 0.02 |
| | (Propyl hydroxy | | | |
| | benzoate) | | | |
| 10. | Lavender oil | Penetration | 40.0 | 4.0 |
| | | enhancer and | | |
| | | Fragrant | | |

WATER PHASE

| | | | | |
|---|---|---|---|---|
| 11. | Citric acid anhydrous | Moisturizing and | 1.30 | 0.13 |
| | | buffering agent | | |
| 12. | Purified water | A topical carrier | 305.32 | 30.53 |
| 13. | Purified water | | 405.05 | 40.51 |
| | (Part II)* | | | |

### Example 5

Preparation of Eflornithine hydrochloride cream composition in the present invention:

| | | | | |
|---|---|---|---|---|
| Sr No. | Ingredients | Function | mg/g | % w/w |

DRUG PHASE

| | | | | |
|---|---|---|---|---|
| 1. | Eflornithine HCl | Active | 100.0 | 10.0 |
| | monohydrate | | | |

OIL PHASE

| | | | | |
|---|---|---|---|---|
| 2. | Glycerylstearate and | Oil in Water | 50.0 | 5.0 |
| | Macrogol stearate | Emulsifier | | |
| | (Arlacel ™ 165) | | | |
| 3. | Cetearyl alcohol and | Emulsion | 75.03 | 7.50 |
| | Macrogol cetostearyl | stabiliser | | |
| | ether (Cosmowax ™ D) | | | |
| 4. | Light liquid paraffin | Vehicle | 30.0 | 3.0 |
| 5. | Stearyl alcohol | Emulsifier | 20.0 | 2.0 |
| 6. | Polydimethylsiloxane | Defoaming agent | 20.0 | 2.0 |
| | (Silicone fluid) or | | | |
| | Dimethicone | | | |
| 7. | Citric acid anhydrous | Moisturizing and | 1.30 | 0.13 |
| | | buffering agent | | |
| 8. | Methyl paraben | Preservative | 1.80 | 0.18 |
| | (Methyl hydroxy | | | |
| | benzoate) | | | |
| 9. | Propyl paraben | Preservative | 0.2 | 0.02 |
| | (Propyl hydroxy | | | |
| | benzoate) | | | |
| 10. | Lavender oil | Penetration | 40.0 | 4.0 |
| | | enhancer and | | |
| | | Fragrant | | |

WATER PHASE

| | | | | |
|---|---|---|---|---|
| 11. | Citric acid anhydrous | Moisturizing and | 1.30 | 0.13 |
| | | buffering agent | | |
| 12. | Purified water | A topical carrier | 305.32 | 30.53 |
| 13. | Purified water | | 355.05 | 35.51 |
| | (Part II)* | | | |

### Example 6

Preparation of Eflornithine hydrochloride cream composition in the present invention:

| | | | | |
|---|---|---|---|---|
| Sr No. | Ingredients | Function | mg/g | % w/w |

DRUG PHASE

| | | | | |
|---|---|---|---|---|
| 1. | Eflornithine HCl | Active | 130.9 | 13.9 |
| | monohydrate | | | |

OIL PHASE

| | | | | |
|---|---|---|---|---|
| 2. | Glycerylstearate and | Oil in Water | 50.0 | 5.0 |
| | Macrogol stearate | Emulsifier | | |
| | (Arlacel ™ 165) | | | |
| 3. | Cetearyl alcohol and | Emulsion | 75.03 | 7.50 |
| | Macrogol cetostearyl | stabiliser | | |
| | ether (Cosmowax ™ D) | | | |
| 4. | Light liquid paraffin | Vehicle | 30.0 | 3.0 |
| 5. | Stearyl alcohol | Emulsifier | 20.0 | 2.0 |
| 6. | Polydimethylsiloxane | Defoaming agent | 20.0 | 2.0 |
| | (Silicone fluid) or | | | |
| | Dimethicone | | | |
| 7. | Citric acid anhydrous | Moisturizing and | 1.30 | 0.13 |
| | | buffering agent | | |
| 8. | Methyl paraben | Preservative | 1.80 | 0.18 |
| | (Methyl hydroxy | | | |
| | benzoate) | | | |
| 9. | Propyl paraben | Preservative | 0.2 | 0.02 |
| | (Propyl hydroxy | | | |
| | benzoate) | | | |
| 10. | Lavender oil | Penetration | 1.0 | 0.1 |
| | | enhancer and | | |
| | | Fragrant | | |

WATER PHASE

| | | | | |
|---|---|---|---|---|
| 11. | Citric acid anhydrous | Moisturizing and | 1.30 | 0.13 |
| | | buffering agent | | |
| 12. | Purified water | A topical carrier | 305.32 | 30.53 |
| 13. | Purified water | | 363.15 | 35.51 |
| | (Part II)* | | | |

### Example 7

Preparation of Eflornithine hydrochloride cream composition in the present invention:

| | | | | |
|---|---|---|---|---|
| Sr No. | Ingredients | Function | mg/g | % w/w |

DRUG PHASE

| | | | | |
|---|---|---|---|---|
| 1. | Eflornithine HCl | Active | 130.9 | 13.9 |
| | monohydrate | | | |

OIL PHASE

| | | | | |
|---|---|---|---|---|
| 2. | Glycerylstearate and | Oil in Water | 50.0 | 5.0 |
| | Macrogol stearate | Emulsifier | | |
| | (Arlacel ™ 165) | | | |
| 3. | Cetearyl alcohol and | Emulsion | 75.03 | 7.50 |
| | Macrogol cetostearyl | stabiliser | | |
| | ether (Cosmowax ™ D) | | | |
| 4. | Light liquid paraffin | Vehicle | 30.0 | 3.0 |
| 5. | Stearyl alcohol | Emulsifier | 20.0 | 2.0 |
| 6. | Polydimethylsiloxane | Defoaming agent | 20.0 | 2.0 |
| | (Silicone fluid) or | | | |
| | Dimethicone | | | |
| 7. | Citric acid anhydrous | Moisturizing and | 1.30 | 0.13 |
| | | buffering agent | | |
| 8. | Methyl paraben | Preservative | 1.80 | 0.18 |
| | (Methyl hydroxy | | | |
| | benzoate) | | | |
| 9. | Propyl paraben | Preservative | 0.2 | 0.02 |
| | (Propyl hydroxy | | | |
| | benzoate) | | | |
| 10. | Lavender oil | Penetration | 40.0 | 4.0 |
| | | enhancer and | | |
| | | Fragrant | | |

WATER PHASE

| | | | | |
|---|---|---|---|---|
| 11. | Citric acid anhydrous | Moisturizing and | 1.30 | 0.13 |
| | | buffering agent | | |
| 12. | Purified water | A topical carrier | 305.32 | 30.53 |
| 13. | Purified water | | 324.15 | 31.61 |
| | (Part II)* | | | |

### Example 8

Preparation of Eflornithine hydrochloride cream composition in the present invention:

| | | | | |
|---|---|---|---|---|
| Sr No. | Ingredients | Function | mg/g | % w/w |

DRUG PHASE

| | | | | |
|---|---|---|---|---|
| 1. | Eflornithine HCl | Active | 130.9 | 13.9 |
| | monohydrate | | | |

OIL PHASE

| | | | | |
|---|---|---|---|---|
| 2. | Ceteareth-20 | Oil in Water | — | 5.0 |
| | | Emulsifier | | |
| 3. | Cetearyl alcohol | Emulsion | 75.03 | 7.50 |
| | | stabiliser | | |
| 4. | Stearyl alcohol | Emulsifier | 20.0 | 2.0 |
| 5. | Dimethicone | Defoaming agent | 20.0 | 2.0 |
| 6. | Glyceryl stearate and | Moisturizing and | 1.30 | 0.13 |
| | PEG-100 stearate | buffering agent | | |
| 7. | Methyl paraben | Preservative | 1.80 | 0.18 |
| | (Methyl hydroxy | | | |
| | benzoate) | | | |
| 8. | Propyl paraben | Preservative | 0.2 | 0.02 |
| | (Propyl hydroxy | | | |
| | benzoate) | | | |
| 9. | Phenoxyethanol | | | |
| 10. | Mineral oil | Moisturising | 40.0 | 4.0 |
| | | agent | | |

WATER PHASE

| | | | | |
|---|---|---|---|---|
| 11. | Purified water | A topical carrier | 305.32 | 30.53 |
| 12. | Purified water | | 405.45 | 34.74 |
| | (Part II)* | | | |

### Preparation Method of Eflornithine Hydrochloride Cream 13.9% and Lavender Oil

### 1: Procedure for Preparation of Oil Phase

The oil phase is prepared by transferring glyceryl stearate and macrogol stearate, cetearyl alcohol and macrogol ceto stearyl ether, light liquid paraffin, stearyl alcohol and poly dimethyl siloxane in jacketed oil phase vessel, melted the oil phase materials by maintaining the temperature at 70° C. to 80° C. Methyl paraben and propyl paraben are added to the melted mass by continuous stirring at a temperature 50° C. to 90° C. The temperature is maintained at 70° C. to 80° C. with continuous stirring. Lavender oil is added slowly into the melted mass. The temperature of the molten mass is maintained at constant temperature range of 70° C. to 80° C.

### 2: Procedure for Preparation of Aqueous Phase

Exactly half dispensed quantity of Purified Water was transferred in to Glass beaker and kept on magnetic stirrer for heating at the temperature of 60° C-65° C. Citric acid is added in the Purified water with continuous stirring to get clear solution.

### Addition of Oil Phase to Aqueous Phase

The oil phase material is added to the aqueous phase by maintaining the temperature to 70° C-80° C.

### 3. Procedure for Preparation of Drug Phase

The remaining half quantity of purified water was transferred in to the glass beaker and purified water was heated at the temperature of 55° C-60° C. Eflornithine hydrochloride monohydrate is added to purified water with continuous stirring to get clear solution forming a drug solution phase.

### 4. Drug Phase Addition to Oil/Water Phase Preparation

The drug solution phase is then added to oil-in-water phase with continuous stirring by maintaining the temperature to 40° C. to 80° C. and homogenize for 30 minutes. Cooled to room temperature with continuous stirring till the cream congeals. The pH is maintained to 3 to 5. Additional quantity of purified water is added till the desired weight of bulk quantity of cream is achieved. Cooled to room temperature with continuous stirring till the cream congeals and filled in lami tubes which are filled with 30 gm of eflornithine hydrochloride cream using automatic tube filling and sealing machine.

### Efficacy Study on Rats

### Procedure for Efficacy Study on Mice

In the present invention, animal study was conducted for assessing the pharmacodynamics behaviour of eflornithine hydrochloride cream with penetration enhancer as lavender oil on mice. The animal study is carried out with (8-10 weeks old), C57BL/6 mice. The animals were housed 3 per cage in polypropylene cages with autoclaved corn cob as bedding material and the general environmental conditions were strictly controlled.

There are about 15 mice which were divided into 5 experimental groups (three mice per group). Waxing was done on the lower dorsal skin of anesthetized mice. After two hours, the waxed area of the skin was treated with the cream samples of each formulation (50 mg per mouse per treatment) as per below given table:

| Group | Composition | Eflornithine (% w/w) | Lavender oil (% w/w) |
|---|---|---|---|
| 1. | Formulation 1 | 0 | 4.0 |
| 2. | Formulation 2 | 3.0 | 0.1 |
| 3. | Formulation 3 | 3.0 | 4.0 |
| 4. | Formulation 4 | 5.0 | 4.0 |
| 5. | Formulation 5 | 10.0 | 4.0 |
| 6. | Formulation 6 | 13.9 | 0.1 |
| 7. | Formulation 7 | 13.9 | 4.0 |
| 8. | Formulation 8 | 13.9 | — |
| | (Vaniqa ®) | | |
| 9. | Control | — | — |

For repeat dose daily administration, the cream samples was applied by using a butter paper having size of 3 x 3 inch (by rubbing on waxed area of skin with butter paper) 2 times a day at an interval of at least 8 hours. In control group containing 3 mice, the hair was removed by waxing method, but the area will not be treated by application with the cream. The hair re-growth in each group of mice was evaluated by taking digital photographs with high resolution (1920×1080) of the mouse skin areas daily.

### Results for Efficacy Study on Mice:

In the present invention, the animal studies were conducted with the application of cream formulations comprising Eflornithine Hydrochloride and Lavender oil on the lower dorsal skin of anesthetized mice by which the hair area was removed by waxing method, having different concentrations of eflornithine (0%, 3%, 5%, 10% and 13.9%) and with different concentrations of lavender oil (0.1% and 4%) and hair growth was observed on 5^{th} day, 11^{th} day and 18^{th} day.

On zero day, hair in the lower dorsal skin of mice were removed by waxing, at the end of 5^{th} day, 11^{th} day and 18^{th} day animals of each experimental group were photographed with digital camera under anaesthesia to evaluate hair growth. Mice of group 1 was treated with only lavender oil, mice of group 2 to 7 were treated with eflornithine (varying concentration 3% to 13.9%) and lavender oil (varying concentration 0.1% to 4%), mice in group 8 was treated with marketed formulation and mice in group 9 were controlled group i.e. received no treatment after hair removal. The observations are mentioned below

At 5^{th} Day: Till 5^{th} day controlled group as well as treatment groups (with test formulation 1 to 8 and Vaniqa®) showed comparable hairless skin suggesting natural lag of significantly visible hair growth after hair removal.

At 11^{th} Day: At day 11 control group showed sufficiently visible hair growth. Group 1 and 2 showed hair growth lesser than compared to control group but higher than compared to other groups suggesting lavender oil alone at concentration of 4% (Group 1) and eflornithine in low concentration (3%) in combination with low concentration of lavender oil (0.1%) is not sufficient to show prolonged effect on reduction of hair growth. Hair growth reduction in group 6 (Eflornithine 13.9% +lavender oil 0.1%) and group 8 (Vaniqa®) were comparable and better than control group suggesting effectiveness of higher concentration of eflornithine alone or in combination with lower quantity of lavender oil in hair growth reduction. Group 3 to 7 having varying concentration of eflronithine (from 3% to 13.9%) with constant concentration of lavender oil 4% showed very less to no hair growth suggesting better control of eflornithine with 4% lavender oil concentration in reduction of hair growth. Group 3 to 5 had eflornithine concentration lower than the marketed formulation yet the reduction in hair growth was comparable suggesting effectiveness of combination of eflorntihine at lower than marketed concentration when combined with lavender oil 4%.

At 18^{th} Day: At day 18 all the groups other than group 7 (Eflornithine 13.9%+Lavender Oil 4%) showed significant hair growth. Suggesting superiority of Eflornithine 13.9%+Lavender Oil 4% combination over existing marketed formulation.

### Conclusion for Efficacy Study on Mice:

The results showed that Eflornithine hydrochloride cream with penetration enhancer as lavender oil is effective with the application of two times a day at an interval of at least 8 hours in the animal study carried out in C57BL6 mice.

The hair growth inhibitory activity of eflornithine was enhanced when the eflornithine cream treated with penetration enhancer as lavender oil was applied onto a waxed mice skin area as confirmed in the animal study.

The cream composition of the present invention which includes Eflornithine hydrochloride with lavender oil as penetration enhancer is effective for the hair growth retardation than the marketed composition named Vaniqa®. The formulation comprising eflornithine is 13.9% and lavender oil is 4.0% has markedly inhibited the hair growth in mice on 18^{th} day. However, when the mice treated with Vaniqa®; slight growth of hair was observed on 11^{th} day and by 18^{th} day, full hair growth was observed in the mice.

Thus the integration of treating lavender oil into topical eflornithine composition represents a potentially viable approach to increase eflornithine's ability to inhibit hair growth.

1. A topical cream composition, effective to reduce the rate of skin hair growth, wherein the composition comprises:
a) eflornithine, a pharmaceutically acceptable salt thereof, or a prodrug thereof at a concentration of about 3% w/w to 13.9% w/w of total composition,
b) a penetration enhancer at a concentration of about 0.1% w/w to about 25.0% w/w of total composition,
c) an oil in water emulsifier at a concentration of about 1.0% w/w to about 12.0% w/w of total composition,
d) an emulsion stabiliser at a concentration of about 3.75% w/w to about 12.0% w/w of total composition,
e) an emulsifier at a concentration of about 1.0% w/w to about 12.0% w/w of total composition,
f) a defoaming agent at a concentration of about 1.0% w/w to about 9.0% w/w of total composition,
g) a moisturizing and/or buffering agent at a concentration of about 0.01% w/w to about 2.0% w/w of total composition,
h) a preservative at a concentration of about 0.1% w/w to about 0.8% w/w of total composition and
i) a topical carrier.

2. The topical composition as claimed in claim 1, wherein the composition comprises a pharmaceutically acceptable salt of eflomithine, said salt being eflomithine hydrochloride.

3. (canceled)

4. The topical composition as claimed in claim 14, wherein the penetration enhancer is lavender oil.

5. The topical composition as claimed in claim 15, wherein the oil in water emulsifier is selected from the group consisting of glyceryl stearate and macrogol stearate.

6. The topical composition as claimed in claim 16, wherein the emulsion stabiliser is selected from the group consisting of cetostearyl alcohol and macrogol cetostearyl ether.

7. The topical composition as claimed in claim 15, wherein the emulsifier is stearyl alcohol.

8. The topical composition as claimed in claim 17, wherein the defoaming agent is polydimethylsiloxane.

9. The topical composition as claimed in claim 18, wherein the moisturizing and/or buffering agent is citric acid anhydrous.

10. The topical composition as claimed in claim 19, wherein the preservative is methyl paraben or propyl paraben.

11. The topical composition as claimed in claim 1, wherein the composition is an oil in water emulsion based cream.

12. A process for preparing a topical composition of eflornithine, a pharmaceutically acceptable salt thereof, or a prodrug thereof, comprising the steps of:
a) preparing an oil phase by transferring glyceryl stearate and macrogol stearate, ceteostearyl alcohol and macrogol ceto stearyl ether, light liquid paraffin, stearyl alcohol and poly dimethyl siloxane in jacketed oil phase vessel,
b) adding methyl paraben and propyl paraben to the melted mass by continuous stirring at a temperature of 50° C. to 90° C.,
c) adding lavender oil slowly into the melted mass by continuous stirring,
d) preparing an aqueous phase by transferring exactly half dispensed quantity of purified water and heating to a temperature of 60° C. to 65° C. and citric acid is added with continuous stirring to get a clear solution,
e) adding the oil phase to the aqueous phase by maintaining the temperature to 70° C. to 80° C. to obtain an oil-in-water phase,
f) Preparing a drug solution phase by adding eflornithine hydrochloride monohydrate to the remaining half-quantity of purified water with continuous stirring,
g) Adding the drug solution phase to the oil-in-water phase with continuous stirring by maintaining the temperature to 40° C. to 80° C. to produce a mixture, and
h) Cooling the mixture of step (g) to room temperature with continuous stirring till the mixture congeals to produce the topical composition of eflornithine as a cream, and maintaining the pH to 2 to 7.

13. (canceled)

14. The topical composition as claimed in claim 1, wherein the penetration enhancer is selected from the group consisting of angelica oil, lavender oil, cyperus oil, cinnamon oil, basil oil, jasmine oil, clove oil, and a mixture thereof.

15. The topical composition as claimed in claim 1, wherein:
the oil in water emulsifier is selected from the group consisting of glyceryl stearate, macrogol stearate, sorbitan stearate, poly glyceryl oleate, lecithin, sorbitan monooleate, lanolin, and a mixture thereof and
the emulsifier is selected from the group consisting of stearyl alcohol, tragacanth, sodium lauryl sulphate, sodium dioctylsulfosuccinate, and a mixture thereof.

16. The topical composition as claimed in claim 1, wherein the emulsion stabiliser is selected from the group consisting of cetostearyl alcohol, macrogol cetostearyl ether, soy lecithin, sodium phosphate, mono and diglycerides, sodium stearoyl lactylate, and a mixture thereof.

17. The topical composition as claimed in claim 1, wherein the defoaming agent is selected from the group consisting of polydimethyl siloxane, sulfonated oils, mineral oils, and a mixture thereof.

18. The topical composition as claimed in claim 1, wherein the moisturizing and/or buffering agent is selected from the group consisting of glycerine, citric acid anhydrous, phosphoric acid, lactic acid, glycolic acid, citric acid, malic acid, tartaric acid, gluconic acid, gluconolactone sodium hydroxide, monobasic sodium phosphate, and a mixture thereof.

19. The topical composition as claimed in claim 1, wherein the preservative is selected from the group consisting of methyl paraben, propyl paraben, phenoxyethanol, ethylhexylglycerin, propylene glycol, and a mixture thereof.

20. The topical composition as claimed in claim 1, wherein the topical carrier is purified water or light liquid paraffin.

21. The process for preparing a topical composition as claimed in claim 13, further comprising filling a tube with the cream obtained in step (h) using an automatic tube filling and sealing machine.
